(19) United States
(12) Patent Application Publication
(10) Pub. No.: US 2024/0112139 A1
(43) Pub. Date: Apr. 4, 2024

(54) PRESENCE INFORMATION MANAGEMENT SYSTEM AND PRESENCE INFORMATION MANAGEMENT METHOD

(71) Applicant: Panasonic Intellectual Property Management Co., Ltd., Osaka (JP)

(72) Inventors: Sonoko HIRASAWA, Kanagawa (JP); Noriyuki KUGOU, Kanagawa (JP)

(73) Assignee: Panasonic Intellectual Property Management Co., Ltd., Osaka (JP)

(21) Appl. No.: 18/268,479

(22) PCT Filed: Oct. 5, 2021
(86) PCT No.: PCT/JP2021/036824

§ 371 Date: Jun. 20, 2023

(30) Foreign Application Priority Data
Dec. 21, 2020 (JP) 2020-211007

(51) Int. Cl. G06Q 10/10; A61B 5/00; A61B 5/01; G06V 20/52; G06V 40/10; G06V 40/16; G16H 40/67

(52) U.S. Cl. CPC G06Q 10/10; A61B 5/01; A61B 5/6887; G06V 20/52; G06V 40/103; G06V 40/166; G06V 40/172; G16H 40/67

(57) ABSTRACT

A system and a method for managing presence information on a person's presence in a free address office, which can be updated and adjusted with less time and effort for adaptation to a change in the office layout, and can efficiently collect information about the person's health. The system includes an in-room camera and an information collection robot. Upon detection of a seated person, the robot moves toward an area where the seated person is detected and captures a face image of the seated person with a face camera therein. Then, the system performs a face verification operation based on the face image to identify the seated person and generates the person's presence information. The system may also generate information on whether a person is wearing a mask or not, and/or acquire a person's vital information from a vital sensor in the robot.

### TECHNICAL FIELD

The present invention relates to a presence information management system and a presence information management method, in which a processing controller manages presence information on a person's presence in an office area.

### BACKGROUND ART

In recent years, free address offices, in which users can freely select office seats to work, have been drawing attention from the viewpoint of activating communication between workers and encouraging collaboration across different departments and divisions.

In the case of such a free address office, as a visitor to the office does not know the seating position of a person the visitor wants to meet with, it would take some time for the visitor to locate the person in the office. Known technologies that address this problem include a system for presenting a guidance indicating who is seated at where in the office to a visitor (Patent Document 1).

### PRIOR ART DOCUMENT (S)

### Patent Document(s)

Patent Document 1: JP2019-144918A

### SUMMARY OF THE INVENTION

### Task to be Accomplished by the Invention

An omnidirectional camera installed on the ceiling of an office makes it possible to detect whether or not a person is present in the office based on an image captured by the camera. However, images captured by the omnidirectional camera cannot be used to perform a face verification operation, i.e., to verify identity of a seated person in an office based on a face image of the seated person. Thus, an additional system or device is required to verify identity of a seated person.

Some systems of the prior art are configured to identify the seating position of each person who has entered a free address area, by using various identification technologies, such as installing a tag detector at each seat to detect a wireless tag carried by a seated person, installing a camera at each seat to capture a face image of a seated person, installing a card reader at each seat to read an IC card carried by a seated person, or providing a seat map screen on which a person can operate to enter the person's seating position.

However, when such a system for identifications of a seated person is used, there is a problem that, every time a change in the office layout occurs, the system requires update and adjustment for adaptation to the change, which takes a lot of time and effort.

From the perspective of preventing the spread of infectious diseases such as new coronavirus infection (COVID-19), there is a need for a system capable of collecting health-related information on a person who has entered the office; such as information on the person's health care (e.g., whether a person is wearing a mask or no mask) and information on the person's state of health (a measured body temperature). In this case, when such a system is configured to collect health-related information on a person's state of health concurrently with collecting the person's presence information, it will become possible to efficiently collect health-related information.

The present invention has been made in view of such problems of the prior art, and a primary object of the present invention is to provide a presence information management system and a presence information management method for managing presence information on a person's presence in a free address office, which can be updated and adjusted with less time and effort for adaptation to a change in the office layout or other changes, and which also can efficiently collect information on the person's health.

### Means to Accomplish the Task

An aspect of the present invention provides a presence information management system, in which a processing controller manages presence information on a person's presence in an office area, the system comprising: an in-area camera for capturing an area image of an area within the office area; and an information collection robot configured to move within the office area, wherein the information collection robot comprises a face camera for capturing a face image of a seated person in a presence detection area in the office area, wherein, when detecting a seated person in a presence detection area based on an area image captured by the in-area camera, the processing controller controls the information collection robot such that the information collection robot moves to a location near the presence detection area where the seated person has been detected and captures a face image of the seated person with the face camera, and wherein, when the face image is captured, the processing controller performs a face verification operation, aiming to verify identity of the seated person, based on the face image captured by the face camera, and when the face verification operation successfully completes, the processing controller generates the presence information associated with the seated person.

Another aspect of the present invention provides a presence information management method in which a processing controller manages presence information on a person's presence in an office area, the method comprising: detecting a seated person in a presence detection area based on an area image captured by an in-area camera; upon the detection, controlling an information collection robot provided with a face camera such that the information collection robot moves to a location near a presence detection area where the seated person has been detected and captures a face image of the seated person with the face camera; and performing a face verification operation, aiming to verify identity of the seated person, based on the face image captured by the face camera, and when the face verification operation successfully completes, generating the presence information associated with the seated person.

### Effect of the Invention

According to the present invention, a system includes an information collection robot which can freely move within an office area for identification of seated persons. This enables implementation of systems which can be updated and adjusted with less time and effort for adaptation to a change in the office layout or other changes in the office area.

### BRIEF DESCRIPTION OF THE DRAWINGS

FIG. 1 is a diagram showing an overall configuration of a presence information management system according to a first embodiment of the present invention;

FIG. 2 is an explanatory diagram showing a layout plan of an office, an arrangement of in-room cameras 1, and presence detection areas in the office of the first embodiment;

FIG. 3 is a diagram showing a schematic configuration of an information collection robot 2 of the first embodiment;

FIG. 4 is an explanatory diagram showing an outline of processing operations performed by a presence management server 3 and a robot control server 4 of the first embodiment;

FIG. 5 is a block diagram showing schematic configurations of the presence management server 3 and the robot control server 4 of the first embodiment;

FIG. 6 is an explanatory diagram showing a presence status confirmation screen in a map mode displayed on a user terminal 5 of the first embodiment;

FIG. 7 is an explanatory diagram showing a presence status confirmation screen in a group mode displayed on a user terminal 5 of the first embodiment;

FIG. 8 is a flow chart showing a procedure of operations performed by the presence management server 3 of the first embodiment;

FIG. 9 is a flow chart showing a procedure of operations performed by the robot control server 4 of the first embodiment;

FIG. 10 is a diagram showing an overall configuration of a presence information management system according to a second embodiment of the present invention;

FIG. 11 is an explanatory diagram showing an arrangement of a first entrance camera 6 and a second entrance camera 7 of the second embodiment;

FIG. 12 is an explanatory diagram showing an outline of processing operations performed by an entry management server 8, a presence management server 3, and a robot control server 4 of the second embodiment;

FIG. 13 is a block diagram showing schematic configurations of the entry management server 8, the presence management server 3, and the robot control server 4 of the second embodiment;

FIG. 14 is a flow chart showing a procedure of operations performed by the entry management server 8 of the second embodiment;

FIG. 15 is a flow chart showing a procedure of operations performed by the presence management server 3 of the second embodiment; and

FIG. 16 is a flow chart showing a procedure of operations performed by the presence management server 3 of the second embodiment.

### DESCRIPTION OF THE PREFERRED EMBODIMENT(S)

A first aspect of the present invention made to achieve the above-described object is a presence information management system, in which a processing controller manages presence information on a person's presence in an office area, the system comprising: an in-area camera for capturing an area image of an area within the office area; and an information collection robot configured to move within the office area, wherein the information collection robot comprises a face camera for capturing a face image of a seated person in a presence detection area in the office area, wherein, when detecting a seated person in a presence detection area based on an area image captured by the in-area camera, the processing controller controls the information collection robot such that the information collection robot moves to a location near the presence detection area where the seated person has been detected and captures a face image of the seated person with the face camera, and wherein, when the face image is captured, the processing controller performs a face verification operation, aiming to verify identity of the seated person, based on the face image captured by the face camera, and when the face verification operation successfully completes, the processing controller generates the presence information associated with the seated person.

According to this configuration, the system includes the information collection robot which can freely move within an office area for identification of seated persons. This enables the system to be updated and adjusted with less time and effort for adaptation to a change in the layout of the office area.

A second aspect of the present invention is the presence information management system of the first aspect, wherein the processing controller generates information as to whether the seated person in the presence detection area is wearing a mask or no mask.

In this configuration, the system can efficiently collect information as to whether the seated person is wearing a mask or no mask as the information on health care of the seated person.

A third aspect of the present invention is the presence information management system of the first aspect, wherein the information collection robot is provided with a vital sensor, and wherein the processing controller causes the information collection robot to measure vital signs of the seated person in the presence detection area by using the vital sensor, thereby acquiring vital information.

In this configuration, the system can efficiently collect vital information on the seated person as information on health care of the seated person. In some cases, the face camera may be a thermo-camera which also serves as a vital sensor.

A fourth aspect of the present invention is the presence information management system of the third aspect, wherein the processing controller acquires at least one of a body temperature and a heart rate as the vital information.

In this configuration, the system can collect a body temperature and/or a heart rate of the seated person as the seated person's vital information.

A fifth aspect of the present invention is the presence information management system of the first aspect, wherein, when detecting a seated person in the presence detection area, the processing controller determines whether or not an absence time period, which is a period of time over which no seated person has been present in the presence detection area before the detection of the seated person, is equal to or less than a predetermined time period, and wherein, when determining that the absence time period is equal to or less than the predetermined time period, the processing controller holds the presence information associated with the detected seated person, and when determining that the absence time period is more than the predetermined time period, the processing controller deletes the presence information associated with the detected seated person.

When a person who was seated in the office and temporarily left the office, returns to the office, this configuration eliminates the need of repeating the step of causing the information collection robot to move toward a presence detection area and performing the face verification operation.

A sixth aspect of the present invention is the presence information management system of the first aspect, wherein the processing controller generates a presence map which indicates a position of each seated person in the office area.

In this configuration, the system enables a user to quickly recognize the presence status (seating status) in the office. In some cases, the presence map may also indicate information on health care and/or the state of health of a seated person.

A seventh aspect of the present invention is a presence information management system, in which a processing controller manages presence information on a person's presence in an office area, the system comprising: an entrance camera for capturing an entrance image of an area in and around an entrance to the office area; an in-area camera for capturing an area image of an area within the office area; and an information collection robot configured to move within the office area, wherein the information collection robot comprises a face camera for capturing a face image of a seated person in a presence detection area in the office area, wherein the processing controller performs a person identification operation on a person who is entering the office area, to thereby identify the person who has entered the office area, wherein, when detecting a seated person in a presence detection area based on an area image captured by the in-area camera, the processing controller performs a person verification operation, aiming to verify identity of the seated person, based on an entrance image captured by the entrance camera and the area image captured by the in-area camera, and when the person verification operation successfully completes, the processing controller generates the presence information associated with the seated person, wherein, when the person verification operation completes in failure, the processing controller controls the information collection robot such that the information collection robot moves to a location near the presence detection area where the seated person has been detected and captures a face image of the seated person with the face camera, and wherein, when the face image is captured, the processing controller performs a face verification operation, aiming to verify identity of the seated person, based on the face image captured by the face camera, and when the face verification operation successfully completes, the processing controller generates the presence information associated with the seated person.

According to this configuration, the system includes the information collection robot which can freely move within an office area for identification of seated persons. This enables the system to be updated and adjusted with less time and effort for adaptation to a change in the layout of the office area. Furthermore, only when the person verification operation completes in failure, the information collection robot is caused to move and capture the face image with the face camera so that the system can perform the face verification operation based on the face image. This enables less frequent use of the information collection robot.

An eighth aspect of the present invention is a presence information management method in which a processing controller manages presence information on a person's presence in an office area, the method comprising: detecting a seated person in a presence detection area based on an area image captured by an in-area camera; upon the detection, controlling an information collection robot provided with a face camera such that the information collection robot moves to a location near a presence detection area where the seated person has been detected and captures a face image of the seated person with the face camera; and performing a face verification operation, aiming to verify identity of the seated person, based on the face image captured by the face camera, and when the face verification operation successfully completes, generating the presence information associated with the seated person.

According to this configuration, the method involves use of the information collection robot which can freely move within an office area for identification of seated persons in the same manner as the first aspect. This enables implementation of systems which can be updated and adjusted with less time and effort for adaptation to a change in the layout of the office area.

A ninth aspect of the present invention is a presence information management method in which a processing controller manages presence information on a person's presence in an office area, the method comprising: performing a person identification operation on a person who is entering the office area, to thereby identify the person who has entered the office area; detecting a seated person in a presence detection area based on an area image captured by an in-area camera; upon the detection, performing a person verification operation, aiming to verify identity of the seated person, based on an entrance image captured by an entrance camera and the area image captured by the in-area camera, and when the person verification operation successfully completes, generating the presence information associated with the seated person; when the person verification operation completes in failure, controlling an information collection robot provided with a face camera such that the information collection robot moves to a location near the presence detection area where the seated person has been detected and captures a face image of the seated person with the face camera; and when the face image is captured, performing a face verification operation, aiming to verify identity of the seated person, based on the face image captured by the face camera, and when the face verification operation successfully completes, generating the presence information associated with the seated person.

According to this configuration, the method involves use of the information collection robot which can freely move within an office area for identification of seated persons in the same manner as the seventh aspect. This enables implementation of systems which can be updated and adjusted with less time and effort for adaptation to a change in the layout of the office area. Furthermore, only when the person verification operation completes in failure, the information collection robot is caused to move and capture the face image with the face camera so that the system can perform the face verification operation based on the face image. This enables less frequent use of the information collection robot.

Embodiments of the present invention will be described below with reference to the drawings.

### First Embodiment

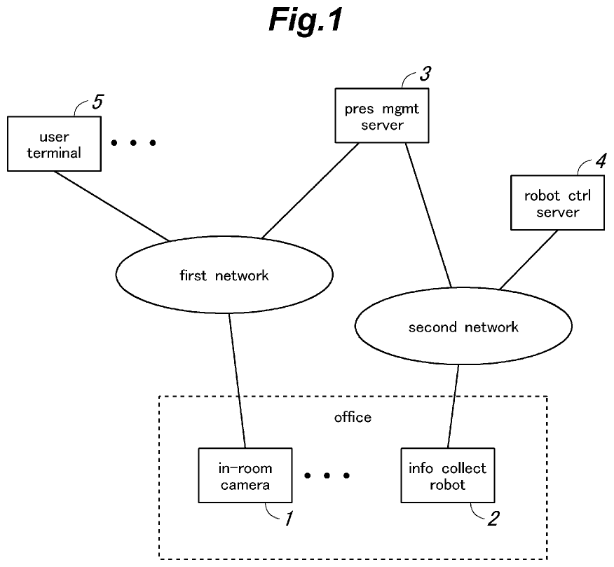

FIG. 1 is a diagram showing an overall configuration of a presence information management system according to a first embodiment of the present invention.

The presence information management system manages presence information on persons' presence in a free address office (office area), and comprises in-room cameras 1 (in-area cameras) for presence detection, an information collection robot 2, a presence management server 3, a robot control server 4, and a user terminal 5 (user device).

The in-room cameras 1, the presence management server 3, and the user terminal 5 are connected to a first network. The information collection robot 2, the presence management server 3, and the robot control server 4 are connected to the second network. The second network provides a communication link through which the information collection robot 2 performs wireless communications. In other embodiments, all the devices may be connected to a single network.

The in-room cameras 1 are installed in the office and capture images of persons present within the office.

The information collection robot 2 is capable of traveling autonomously. Upon receiving instructions from the robot control server 4, the information collection robot 2 moves to a location near a seated person in the office and collects information about the person according to the instructions.

The presence management server 3 performs operations for management of seated persons in the office based on images captured by the in-room cameras 1 and on information collected by the information collection robot 2. In addition, the presence management server 3 also serves as a delivery server and, based on management information stored in a database, delivers presence information on seated persons' presence in the office to the user terminal 5.

The robot control server 4 controls operations of the information collection robot 2.

Based on information provided from the presence management server 3, the user terminal 5 indicates presence information on seated persons' presence in the office to a user.

In other embodiments, the function of managing the database and that of delivering presence information may be implemented in respective independent serves i.e., a database server and a delivery server, instead of being implemented in the presence management server 3.

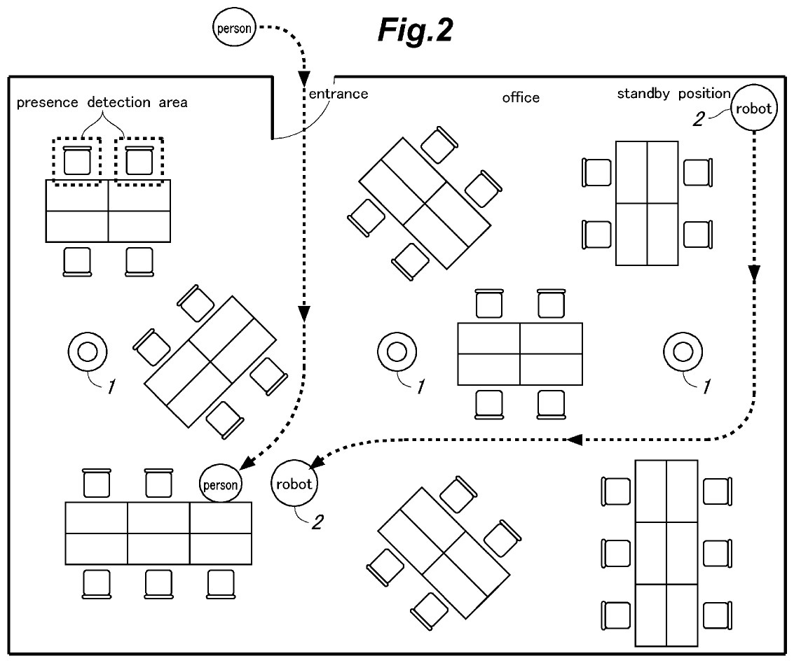

Next, a layout plan of an office, an arrangement of in-room cameras 1, and presence detection areas in the office according to the first embodiment will be described. FIG. 2 is an explanatory diagram showing a layout plan of an office, an arrangement of in-room cameras 1, and presence detection areas in the office.

Desks and office seats (chairs) are arranged side by side in the office. As the office is a free address office, anyone who has entered the office can choose any available office seat and take the seat.

In-room cameras 1 are provided on the ceiling of the office so that the in-room cameras can capture images of persons in the office. Each of the in-room cameras 1 is typically an omnidirectional camera having a fisheye lens and capable of capturing a 360-degree angle of view. In other cases, each in-room camera 1 may be a box camera configured to capture a predetermined range of angles of view. In other cases, each in-room camera 1 may be a simple camera (such as a USB camera) connected to a personal computer(s) in the office.

In the present embodiment, a presence detection area is preset for each office seat in an image captured by each in-room camera 1. The system detects whether or not a person is seated in each office seat based on an image of a corresponding presence detection area. In the example shown in FIG. 2, each presence detection area (in dotted lines) is determined as a rectangle area in a plan view of the office. In practice, however, each presence detection area is determined as a polygonal area in an omnidirectional image (fisheye image) captured by an in-room camera 1.

Each presence detection area is determined at a position where the body of a person seated in a corresponding office seat is expected to be present, and is sized, based on possible sizes of persons' body, so that each presence detection area corresponds to one corresponding person. Typically, in order to preset a presence detection area, a user specifies a scope of a presence detection area in an image captured by an in-room camera 1 and displayed on the screen. However, in some cases, a presence detection area may be preset by detecting some objects (such as seats and desks) in an image captured by an in-room camera 1 and determining a proper area based on the detection results.

The information collection robot 2 is placed in the office. First, the information collection robot 2 stands by at a predetermined standby position in the office, and in response to instructions provided from the robot control server 4, moves to a location near the area where a seated person has been detected in the office, to thereby collect information about the seated person. In the present embodiment, when detecting a seated person in a corresponding presence detection area, the presence management server 3 instructs the robot control server 4 to perform control causing the information collection robot 2 to move to a location near the target person, thereby collecting information on the person.

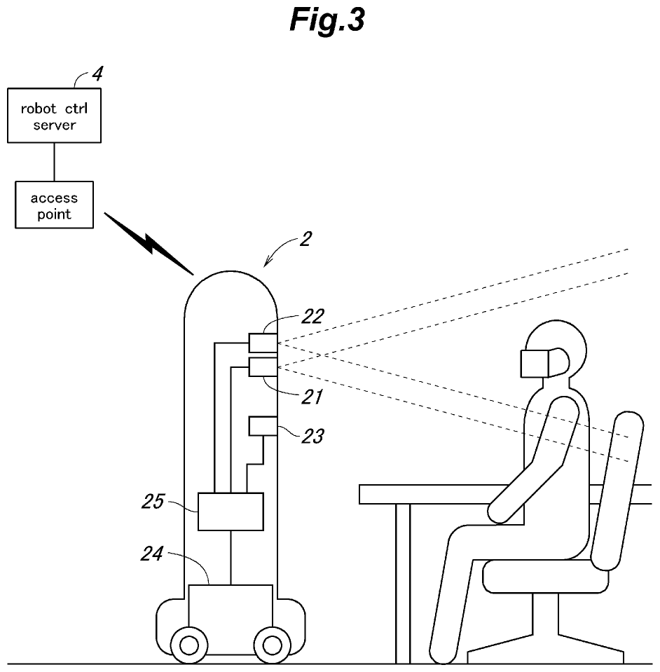

Next, a schematic configuration of an information collection robot 2 of the first embodiment will be described. FIG. 3 is a diagram showing a schematic configuration of the information collection robot 2.

The information collection robot 2 includes a face camera 21, a vital sensor 22, a speaker 23, a travel device 24, and a control device 25.

The face camera 21 captures a face image of a seated person in an office seat. From one or more face images captured by the face camera 21, the system acquires a face image for a face verification operation, aiming to verify identity of the seated person. The face camera 21 is positioned at such a height that the face camera 21 can face the face of a seated person in the office to capture a face image of the seated person from a close distance.

The vital sensor 22 measures vital signs of a seated person in the office in a non-contact manner, to thereby acquire vital information, the vital information including a body temperature and a heart rate (pulse) of the person. An example of the vital sensor 22 is a thermos-camera (infrared camera). In some cases, the face camera 21 may be configured to also function as a thermos-camera capable of providing both a temperature image and a color image so that the face camera 21 can also serve as the vital sensor 22.

The speaker 23 provides audio outputs or voices for various requests, guidance, and notifications in response to instructions from the robot control server 4. For example, the speaker 23 provides a voice that prompts a person to turn the face toward the information collection robot 2 so that the person's face image can be captured from the front. In some cases, the speaker 23 provides audio guidance so that a person can remove hair from the face thereby having the person's body temperature measured properly. The speaker 23 outputs a voice that speaks the name of a person as a notification of a result of the face verification operation and notifies the person of the acquired vital information (e.g., voice that speaks a body temperature). When a person is wearing no mask, the speaker 23 may output a voice that prompts the person to wear a mask. When a dispenser for disinfectant solution is mounted on the information collection robot 2, the speaker 23 may output a voice that prompts a person to disinfect the hands. When a measured body temperature of a person falls within a fever range that indicates the person has a fever (e.g., 37.5 degrees Celsius or higher), the speaker 23 may output a voice that notifies the person of the fever and urges the person to return home.

The travel device 24 includes wheels and motors. The travel device 24 is controlled by the control device 25 and enables the information collection robot 2 to travel autonomously.

The control device 25 includes a communication device for communicating with the robot control server 4, a storage device for storing control programs or other data, and a processing controller for executing the control programs. The processing controller, for example, performs drive control processing operations and makes route planning to avoid obstacles based on images captured by the face camera 21 and detection results of a distance sensor (not shown).

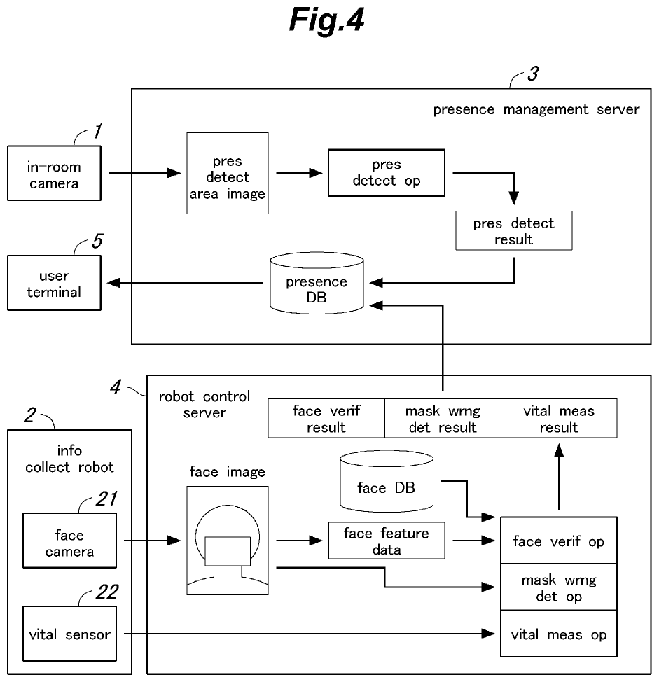

Next, an outline of processing operations performed by the presence management server 3 and the robot control server 4 of the first embodiment will be described. FIG. 4 is an explanatory diagram showing an outline of processing operations performed by the presence management server 3 and the robot control server 4.

The presence management server 3 cuts out a presence detection area (office seat) from an image captured by an in-room camera 1 to thereby acquire a presence detection area image, determines whether or not a person is present in the presence detection area from the presence detection area image, and then sets the presence status of the presence detection area to either present or vacant (absent) based on a result of the determination (presence detection operation). The presence management server 3 periodically carries out the presence detection operation.

When detecting the presence of a person in a presence detection area, the presence management server 3 instructs the robot control server 4 to cause the information collection robot 2 to collect information about the detected person in the presence detection area.

The robot control server 4 controls the operation of the information collection robot 2 in response to an instruction for collecting information provided from the presence management server 3. Specifically, the robot control server 4 performs control causing the information collection robot 2 to move to a location near the target presence detection area, and to capture a face image of a seated person in the presence detection area using the face camera 21.

The robot control server 4 acquires a face image by cutting out a seated person's face from a face image captured by the face camera 21, and extracts face feature data of the seated person from the face image. Then, the robot control server 4 compares the extracted face feature data with face feature data of each person registered in a database for matching, to thereby identify the seated person (face verification operation). Through the face verification operation, the robot control server 4 associates the detected seated person with a registered person, to thereby identify the detected seated person.

The robot control server 4 determines whether a seated person is wearing a mask or no mask based on a face image of the seated person. The robot control server 4 also performs control causing the information collection robot 2 to measure vital signs of a subject person with the vital sensor 22, thereby acquiring vital measurement results as the person's vital information.

The robot control server 4 transmits a face verification result, a result of mask-wearing determination, and measurement results to the presence management server 3 in which the results are registered in a presence database.

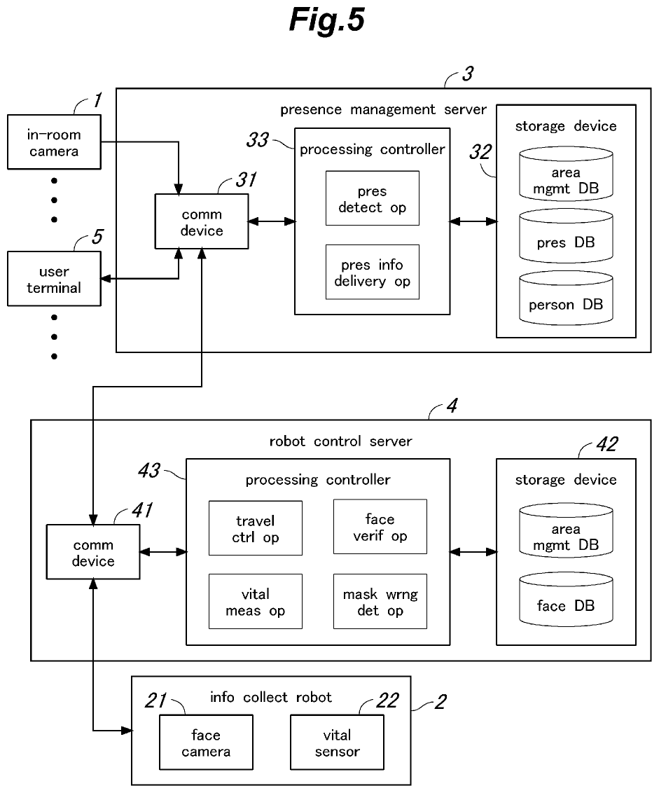

Next, schematic configurations of the presence management server 3 and the robot control server 4 of the first embodiment will be described. FIG. 5 is a block diagram showing schematic configurations of the presence management server 3 and the robot control server 4.

The presence management server 3 includes a communication device 31, a storage device 32, and a processing controller 33.

The communication device 31 communicates with the robot control server 4, instructs the robot control server 4 to cause the information collection robot 2 to move, and receives face verification results, vital measurement results, and results of mask-wearing determination from the robot control server 4. The communication device 31 communicates with the user terminal 5 and transmits presence information on a person's presence in the office to the user terminal 5. The communication device 31 communicates with the in-room cameras 1 and receives captured images from the in-room cameras 1.

The storage device 32 stores programs to be executed by the processing controller 33 and other data. Furthermore, the storage device 32 stores registered information records for an area management database, a presence database, and a person database. The area management database contains registered information records including detection area management information (such as an area ID and position data of each presence detection area). The presence database contains registered information records including, for each presence detection area, an area ID, a presence detection time, and person verification result information (such as a person ID and a person verification score). The person database contains registered information records including person management information (such as a person ID, and person's name, affiliation, and managerial name for each person). Moreover, the storage device 32 temporarily stores images captured by the in-room cameras 1.

The processing controller 33 performs various processing operations by executing programs stored in the storage device 32. In the present embodiment, the processing controller 33 performs the presence detection operation, a presence information delivery operation, and other operations.

In the presence detection operation, the processing controller 33 detects a person in the part of a presence detection area in an image captured by an in-room camera 1, and determines whether or not a person is present in the target presence detection area. When a person is present in the presence detection area, the processing controller 33 sets the presence status of the presence detection area to “present.” When there is no person in the presence detection area, the processing controller 33 sets the presence status of the presence detection area to “vacant (absent).” Then, the processing controller 33 stores presence detection result information (presence detection times, and number of areas) and detailed presence detection result information (presence detection time, area ID, and detected presence status for each area) in the storage device 32.

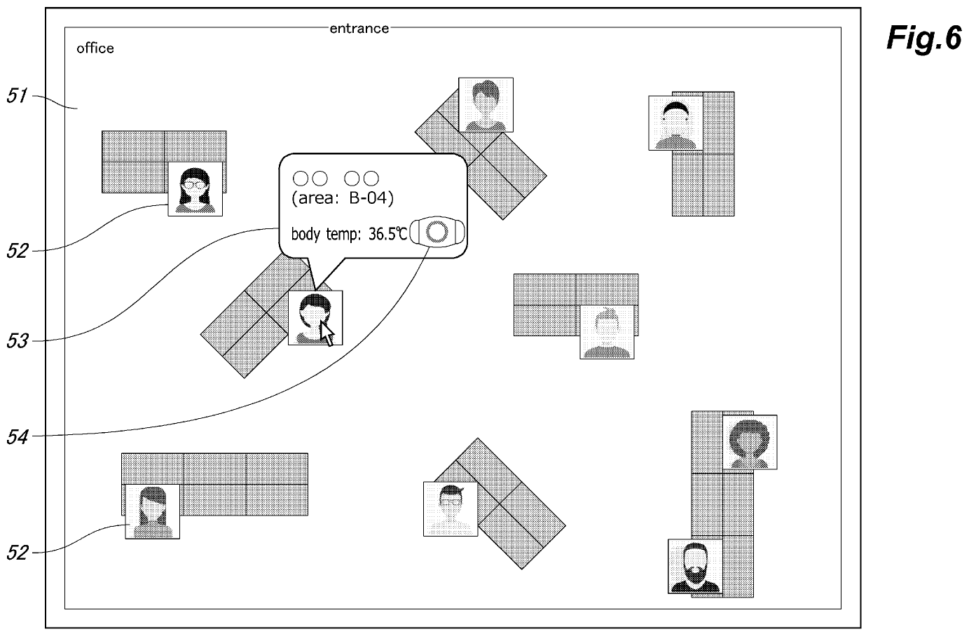

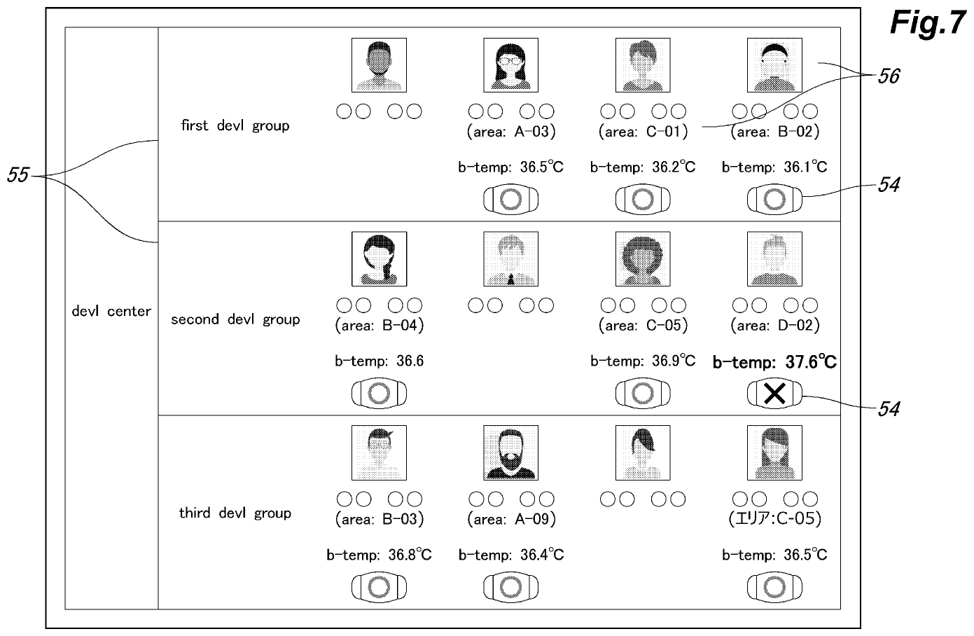

In the presence information delivery operation, the processing controller 33 generates presence information on persons' presence in the office (e.g., position data) based on information records stored in the presence database and the person database, and then delivers the generated presence information to the user terminal 5. In the present embodiment, the processing controller 33 delivers delivered information; that is, information to be displayed on a presence status confirmation screen (see FIGS. 6 and 7) to the user terminal 5.

The robot control server 4 includes a communication device 41, a storage device 42, and a processing controller 43.

The communication device 41 communicates with the information collection robot 2, transmits control information to the information collection robot 2, and receives images captured by the face camera 21 and detection results of the vital sensor 22 from the information collection robot 2. The communication device 41 also communicates with the presence management server 3 and transmits face verification results, vital measurement results, and results of mask-wearing determination acquired by the processing controller 43 to the presence management server 3.

The storage device 42 stores programs to be executed by the processing controller 43 and other data. The storage device 42 also stores registered information records for an area management database, and a face database. The area management database contains registered information records including detection area management information (such as an area ID and position data of each presence detection area). The face database contains face verification information for each person previously registered, specifically, information records such as a person ID and face feature data of each person.

The processing controller 43 performs various processing operations by executing programs stored in the storage device 42. In the present embodiment, the processing controller 43 performs a travel control operation, a face verification operation, a vital measurement operation, and a mask-wearing determination operation and other operations.

In the travel control operation, the processing controller 43 sets a target position at a location near a target presence detection area (i.e., a presence detection area where a person's presence was detected), and performs control causing the information collection robot 2 to move from the standby position to the target position. In the travel control operation, the processing controller 43 also performs control causing the information collection robot 2 to return to the standby position. When multiple persons take seats in succession, resulting in that multiple target positions are to be determined, the processing controller 43 does not necessarily change the target position in order of detection time. For example, the processing controller 43 may perform route optimization to determine an optimal route (shortest route) along which the information collection robot 2 can move to multiple targets positions from one target position to another target position in order of shortest distances from the robot's current position, the route optimization being performed each time the presence detection result is updated.

In the mask-wearing determination operation, the processing controller 43 determines whether a subject person is wearing a mask or no mask based on an image captured by the face camera 21 of the information collection robot 2.

In the face verification operation, the processing controller 43 performs control causing the information collection robot 2 to capture the face of a seated person in the target presence detection area with the face camera 21. Next, the processing controller 43 receives the face image of the seated person captured by the face camera 21 from the information collection robot, and extracts face feature data of the seated person from the received face image. Then, the processing controller 43 acquires face feature data for each person registered in the storage device 42, and compares the face feature data extracted from the face image with the face feature data acquired from the storage device 42 for matching, to thereby identify the seated person in the target presence detection area. When the target seated person is wearing a mask, the processing controller 43 excludes the mask image part in the face image before performing comparison for matching.

In the vital measurement operation, the processing controller 43 performs control causing the information collection robot 2 to measure vital signs (such as a body temperature and a heart rate) of a target person with the vital sensor 22. Then, based on the detection results of the vital sensor 22 received from the information collection robot 2, the processing controller 43 acquires the vital measurement results. In one example, when a body temperature is measured and the face camera 21 is a thermo-camera which can also serve as a vital sensor, the processing controller 43 specifies the position of the forehead in a temperature image captured by the thermos-camera based on the detection result of the face image (color image) captured by the face camera 21 (which also serves as the vital sensor), and acquires a body temperature value at the specified forehead region. The body temperature is not necessarily a temperature at a forehead region or another specific part in the face, and may be a highest epidermal temperature in the face. In some cases, a person's physical condition estimated from the person's facial expression in a face image captured by the face camera 21 may be used as a vital measurement result. In other cases, a person's tension level estimated from the heart rate may be acquired as a vital measurement result.

Next, a presence status confirmation screen displayed on the user terminal 5 of the first embodiment will be described. FIG. 6 is an explanatory diagram showing a presence status confirmation screen in a map mode displayed on the user terminal 5. FIG. 7 is an explanatory diagram showing a presence status confirmation screen in a group mode displayed on the user terminal 5.

The presence management server 3 generates a presence status confirmation screen indicating presence statuses of persons in the office based on information records registered in the database, and delivers the generated screen data to the user terminal 5, which displays the presence status confirmation screen either in a map mode shown in FIG. 6 or in the group mode (list display mode) shown in FIG. 7. A user can switch between the map mode and the group mode by performing a predetermined operation on the screen.

As shown in FIG. 6, the presence status confirmation screen in the map mode (presence map) shows a layout drawing 51 (area map) of the office including office seats. The screen also includes person icons 52, each person icon representing a corresponding seated person, at the respective seats (i.e., persons' positions) in a layout drawing 51 of the office. Each person icon 52 includes a face image of a corresponding person. A person icon 52 may include, in addition to a person's face cut out from a captured image of the person, a portrait of the person or texts such as the person's name.

When a user operates a person icon 52 in the presence status confirmation screen, a word balloon 53 (person information display section) appears in the screen. The word balloon 53 includes detailed information about the person. In the example shown in FIG. 6, the word balloon 53 indicates detailed information about the selected person, which includes the person's name, an ID (area number) of the person's presence detection area (office seat), a measured body temperature of the person, and a mask icon 54 as information on whether or not the person is wearing a mask. The form of a mask icon 54 changes depending on whether a corresponding person is wearing a mask or no mask.

As shown in FIG. 7, the presence status confirmation screen in group mode indicates information about seated persons (present persons) for each of the department groups (e.g., first to third development division groups). More specifically, the presence status confirmation screen includes department group indicating sections 55, and each department group indicating section 5 includes one or more personal data sections 56. A personal data section 56 indicates a person's face image, name, ID (area number) of a presence detection area (office seat), a measured body temperature, and a mask icon 54. In the example shown in FIG. 7, for a person who is not present in the office, a personal data section 56 indicates only the person's face image and name.

When a measured body temperature of a person falls within a fever range (e.g., 37.5° C. or higher) or when the person is wearing no mask, a corresponding personal data section 56 may be highlighted. In the example shown in FIG. 7, a person's measured body temperature is displayed in bold red letters. In other embodiments, a pop-up screen or voice alert may be issued when a measured body temperature of a person falls within the fever range or the person is wearing no mask.

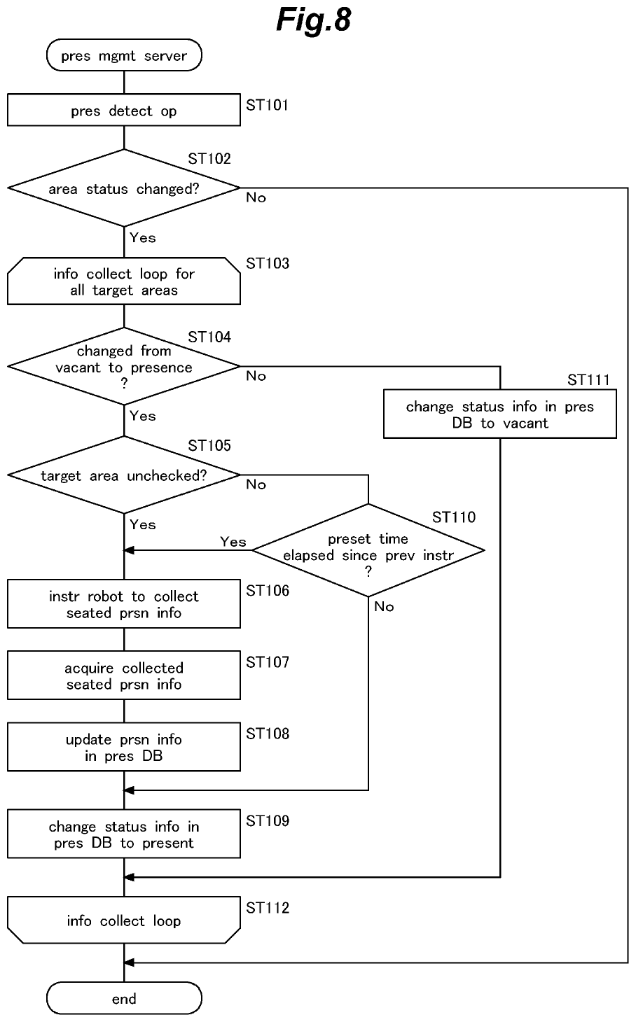

Next, a procedure of operations performed by the presence management server 3 of the first embodiment will be described. FIG. 8 is a flow chart showing a procedure of operations performed by the presence management server 3.

In the presence management server 3, first, the processing controller 33 performs the presence detection operation (ST101).

In the presence detection operation, the processing controller 33 acquires a captured image of an in-room camera 1 and performs a person detection operation on the image of a target presence detection area in the captured image to determine whether or not a person is present in the presence detection area. When determining that a person is present in the target presence detection area, the processing controller 33 sets the presence status of the target presence detection area as “present”, and when determining that no person is present in the target presence detection area, the processing controller 33 sets the presence status of the target presence detection area as “vacant (absent).” Then, the processing controller 33 stores the detection result in the storage device 32. However, in some cases, when detecting belongings of a person, which means that the person is likely to be temporality away from the seat, the processing controller 33 may set the presence status of the target presence detection area as “present” despite detection of no person.

Next, the processing controller 33 determines whether or not the presence status of the presence detection area has changed (ST102).

When the presence status of the presence detection area changes (Yes in ST102), the processing controller 33 performs an information collection operation (information collection loop) using the information collection robot 2, on each presence detection area for which a change in the presence status has been detected (ST103 to ST 112).

In the information collection operation, the processing controller 33 first determines whether or not the presence status of the presence detection area has changed from “vacant (absent)” to “present” (ST104).

When determining that the presence status of the presence detection area has changed from vacant to present, i.e., that a person takes the seat (Yes in ST104), the processing controller 33 determines whether or not the target presence detection area is unchecked, i.e., an instruction for collecting information using the information collection robot 2 has not transmitted to the robot control server 4 (ST105).

When determining that target presence detection area is still unchecked (Yes in ST105), the processing controller 33 transmits an instruction for collecting information from the communication device 31 to the robot control server 4, thereby causing the information collection robot 2 to collect information on a seated person in the target presence detection area (ST106).

Next, the processing controller 33 acquires seated person information; that is, a face verification result, vital measurement results, and a result of mask-wearing determination received at the communication device 31 from the robot control server 4 (ST107).

Next, the processing controller 33 updates person information in the presence database (ST108). Specifically, the processing controller 33 stores the person's ID, vital measurement results, and a result of mask-wearing determination in the presence database as face verification results for the person associated with the target presence detection area. Then, the processing controller 33 updates the information on the presence status of the target area in the presence database from “vacant” to “present” (ST109). Then, the process proceeds to the operation on another presence detection area.

When determining that the target presence detection area is not unchecked, i.e., an instruction for collecting information using the information collection robot 2 has been transmitted to the robot control server 4, (No in ST105), the processing controller 33 determines whether or not more than a predetermined time has elapsed since the previous instruction for collecting information using the information collection robot 2 was transmitted to the robot control server 4 (ST110).

When the processing controller 33 determines that more than the predetermined time has elapsed since the previous instruction for collecting information using the information collection robot 2 was transmitted to the robot control server 4 (Yes in ST110), the process proceeds to ST106 and the processing controller 33 transmits an instruction for collecting information using the information collection robot 2 to the robot control server 4 again. When the processing controller 33 determines that the predetermined time has not yet elapsed since the previous instruction for collecting information using the information collection robot 2 was transmitted to the robot control server 4 (No in ST110), the process proceeds to ST109.

When determining that the presence status of the presence detection area has changed from present to vacant (No in ST104), the processing controller 33 updates the information on the presence status of the target area in the presence database (ST111). Then, the process proceeds to the operation on another presence detection area.

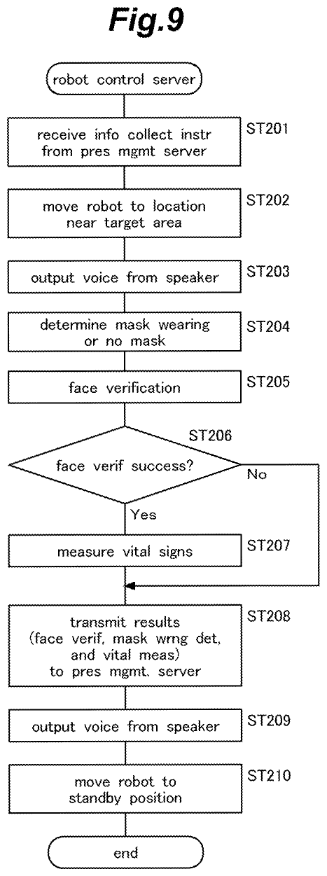

Next, a procedure of operations performed by the robot control server 4 of the first embodiment will be described. FIG. 9 is a flow chart showing a procedure of operations performed by the robot control server 4.

In the robot control server 4, first, the communication device 41 receives an instruction for collecting information using the information collection robot 2 from the presence management server 3 (ST201).

Next, the processing controller 43 sets a target position at a location near the target presence detection area, i.e., the presence detection area where a seated person was detected, and performs control causing the information collection robot 2 to move from the standby position to the target position (ST202).

When the information collection robot 2 reaches the target position, the processing controller 43 then performs control causing the information collection robot 2 outputs a voice from the speaker 23, the voice prompting a seated person to turn the face toward the information collection robot 2 so that the seated person's face image can be captured from the front (ST203).

Next, the processing controller 43 performs a mask-wearing determination operation (ST204). In this operation, the processing controller 43 determines whether the target person is wearing a mask or no mask based on the person's face image captured by the face camera 21 of the information collection robot 2.

Next, the processing controller 43 performs the face verification operation (ST205). In the face verification operation, the processing controller 43 extracts face feature data of the target person from a face image captured by the face camera 21. Then, the processing controller 43 compares the extracted face feature data with face feature data of each person registered in the storage device 42 for matching, to thereby provide a face verification score.

Next, the processing controller 43 determines whether or not the face verification successfully completes, i.e., whether or not the target person is identified as a registered person based on the face verification score (ST206).

When the face verification successfully completes (Yes in ST206), the processing controller 43 performs the vital measurement operation (ST207). The processing controller 43 performs control causing the information collection robot 2 to measure the target person's vital signs with the vital sensor 22 as the target person's vital information, and acquires vital measurement results transmitted from the information collection robot 2.

Next, the processing controller 43 transmits seated person information, i.e., a face verification result, vital measurement results, and a result of mask-wearing determination from the communication device 41 to the presence management server 3 (ST208).

Next, the processing controller 43 performs control causing the information collection robot 2 to output a voice from the speaker 23, the voice notifying the seated person of the completion of the operation for collecting information on the person and results of the operation, i.e., the person's name as a face verification result and vital measurement results (such as a measured body temperature) (ST209).

Next, the processing controller 43 performs control causing the information collection robot 2 to return to the predetermined standby position (ST210).

### Second Embodiment

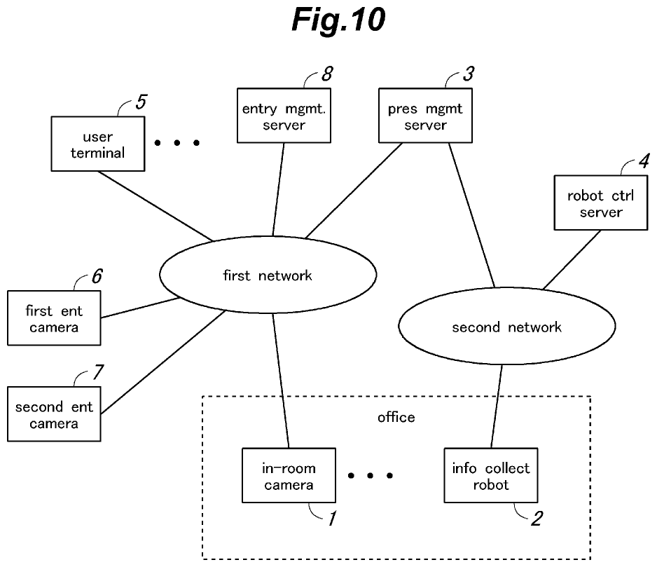

Next, a system according to a second embodiment of the present invention will be described. Except for what will be discussed here, this embodiment is the same as the above-described first embodiment. FIG. 10 is a diagram showing an overall configuration of a presence information management system of the second embodiment.

In the present embodiment, the presence information management system comprises an in-room cameras 1 (in-area camera) for presence detection, an information collection robot 2, a presence management server 3, a robot control server 4, and a user terminal 5 (user device) as in the first embodiment (see FIG. 1), and further comprises a first entrance camera 6 for face verification, a second entrance camera 7 for person verification, and an entry management server 8.

The first entrance camera 6 is installed at or near an entrance of the office and configured to capture an image of the face of a person who is to undergo face verification (face authentication). The second entrance camera 7 is installed at or near the entrance of the office and configured to capture an image of a person who is entering the office (entering person) through the entrance.

The entry management server 8 manages presence information on a person's presence in the office based on an image captured by the first entrance camera 6 and that captured by the second entrance camera 7.

The in-room cameras 1 are installed in the office and capture images of persons present within the office, as in the first embodiment. In the present embodiment, the in-room cameras 1 are also used for person verification, in addition to being used for persons' presence detection as in the first embodiment.

The presence management server 3 performs operations for management of seated persons in the office based on images captured by the in-room cameras 1 and information collected by the information collection robot 2, as in the first embodiment.

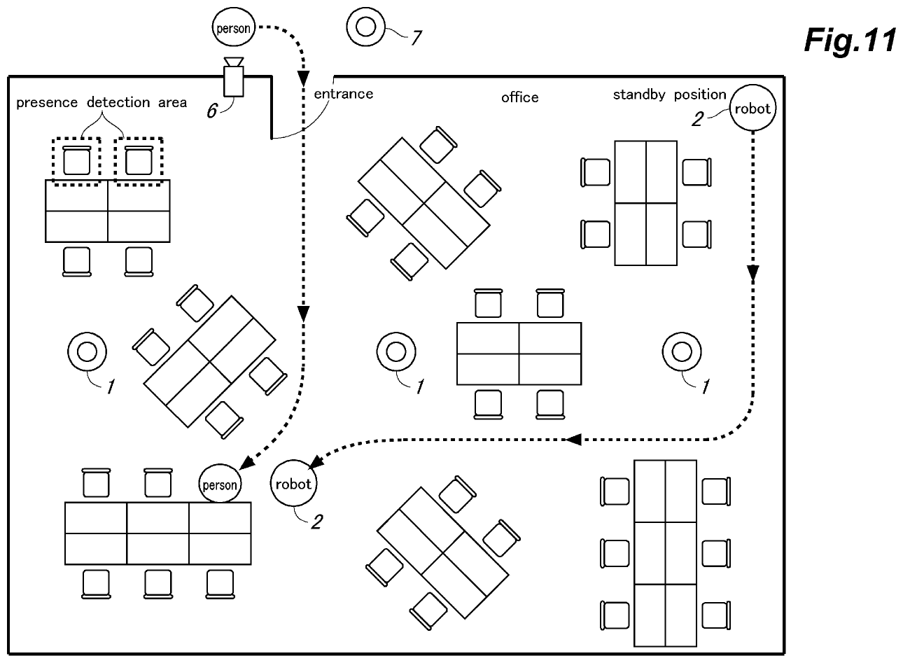

Next, an arrangement of a first entrance camera 6 and a second entrance camera 7 of the second embodiment will be described. FIG. 11 is an explanatory diagram showing an arrangement of the first entrance camera 6 and the second entrance camera 7. A layout plan of the office, an arrangement of in-room cameras 1, and presence detection areas in the office of the second embodiment are the same as those of the first embodiment (see FIG. 2).

A first entrance camera 6 and a second entrance camera 7 are installed at the entrance to the office. The first entrance camera 6 is a box camera configured to capture images within a predetermined angle of view. The first entrance camera 6 captures the face of each person which is entering the office. The second entrance camera 7 is an omnidirectional camera configured to capture 360-degree images using a fisheye lens. The second entrance camera 7 captures the whole body or upper body of a person who is entering the office.

In the example shown in FIG. 2, the second entrance camera 7 is installed outside the office. However, the second entrance camera 7 may be installed inside the office so that the second entrance camera 7 can capture an image of the whole body or upper body of a person entering the office through the entrance.

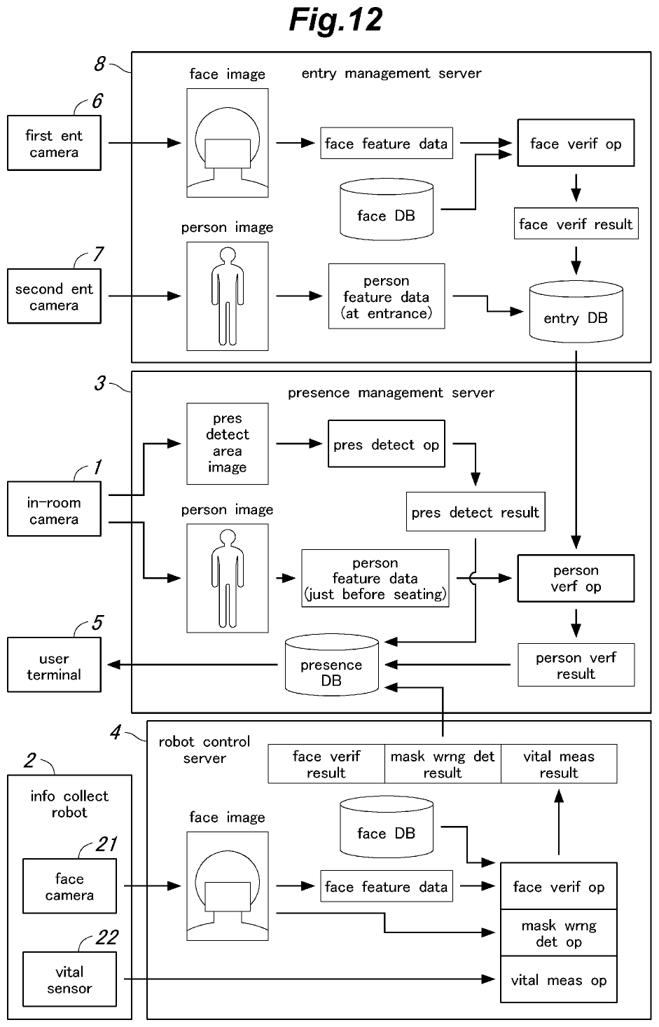

Next, an outline of processing operations performed by an entry management server 8, a presence management server 3, and a robot control server 4 of the second embodiment will be described. FIG. 12 is an explanatory diagram showing an outline of processing operations performed by the entry management server 8, the presence management server 3, and the robot control server 4.

The entry management server 8 cuts out a person's face from an image captured by the first entrance camera 6 to acquire a face image of an entering person (i.e., a person who is entering the office), and extracts face feature data from the acquired face image. Then, the entry management server 8 compares the face feature data of each registered person with the face feature data of the entering person, and identifies the entering person (face verification operation). By performing the face verification operation, the entry management server 8 associates each entering person with a corresponding registered person to thereby identify the entering person. The entry management server 8 registers the person ID of the identified entering person in an entry database therein. When face verification (face authentication) successfully completes for a person, the person can enter the office. In the present embodiment, a person identification operation is performed through face authentication. However, the person identification operation may be performed through card authentication or biometrics authentication.

When face verification successfully completes for an entering person, the entry management server 8 cuts out a part of the whole body or upper body of the entering person from an image captured by the second entrance camera 7 at the same time as the face verification operation, acquires a person image (first person image), and extract, from the person image, person feature data of the entering person (person detection operation). The entry management server 8 registers the person feature data of the entering person in the entry database.

Person feature data represents appearance features of the whole body or upper body of a person, such as colors of the person's clothes, items carried by the person, and the body frame of the person.

In the present embodiment, the system is provided with the first entrance camera 6 for face verification and the second entrance camera 7 for person verification. However, the system may be configured to include a single camera (e.g., an omnidirectional camera) that can be used for both face verification and person verification. When an omnidirectional image captured by an omnidirectional camera is used to acquire a face image for face verification, the system may be configured to convert an omnidirectional image to a panoramic image, and then cut out a part of the face of a person from the panoramic image.

The presence management server 3, as in the first embodiment, cuts out a corresponding presence detection area (office seat) from an image captured by the in-room camera 1 to thereby acquire a presence detection area image, determines whether or not a person is present in the presence detection area from the presence detection area image, and then sets the presence status of the presence detection area to either present or vacant (absent) based on a result of the determination (presence detection operation).

Furthermore, when the presence status of a presence detection area is changed from vacant to present; that is, when a person takes the seat, the presence management server 3 cuts out a part of the whole body or upper body of a person from an image which was captured by an in-room camera 1 immediately before the person takes the seat, to thereby acquire a person image (second person image), and, from the person image, extracts person feature data of the person immediately before taking the seat. Then, the presence management server 3 compares the person feature data of the person immediately before taking the seat with person feature data of each entered person who is registered in the entry database in the entry management server 8 for verification (person verification operation). By performing the person verification operation, the presence management server 3 associates the person who is taking the seat with a corresponding entered person who is registered in the entry database to thereby identify the person who is taking the seat.

When a person is seated on a seat, the person's body is partially hidden by the desk or chair, which can prevent from extraction of proper person feature data. Thus, in the present embodiment, the presence management server 3 acquires a person image from an image captured at the moment when a person is still standing immediately before taking a seat, and extracts person feature data from that person image.

In the first embodiment, the robot control server 4 performs the face verification operation using a face image captured by the face camera 21 of the information collection robot 2 for every detected person. In other words, each time a person is detected in a presence detection area (office seat), the information collection robot 2 is caused to move toward the person and the robot control server 4 performs the face verification operation using the face image captured by the face camera 21. As a result, the information collection robot 2 needs to move more frequently at times of detection of a number of persons (such as at the beginning of a workday).

However, in the present embodiment, the presence management server 3 performs a person verification operation using an image captured by an in-room camera 1, and when the person verification operation completes in failure, the information collection robot 2 is caused to move and capture a face image with the face camera 21 and the robot control server 4 performs the face verification operation based on the face image. When the person verification operation using the acquired image successfully completes, the robot control server 4 does not perform the face verification operation based on a face image captured by the face camera 21 of the information collection robot 2. As a result, the use of the information collection robot 2 becomes less frequent.

In the present embodiment, when the person verification operation using the acquired image compotes in failure, the information collection robot 2 is used for face verification to identify a seated person in a presence detection area (office seat). However, in other embodiments, even when the person verification operation using the acquired image compotes in failure, the information collection robot 2 may be caused to move in order to measure vital signs of the person as vital information and/or determine whether the person is wearing a mask or no mask.

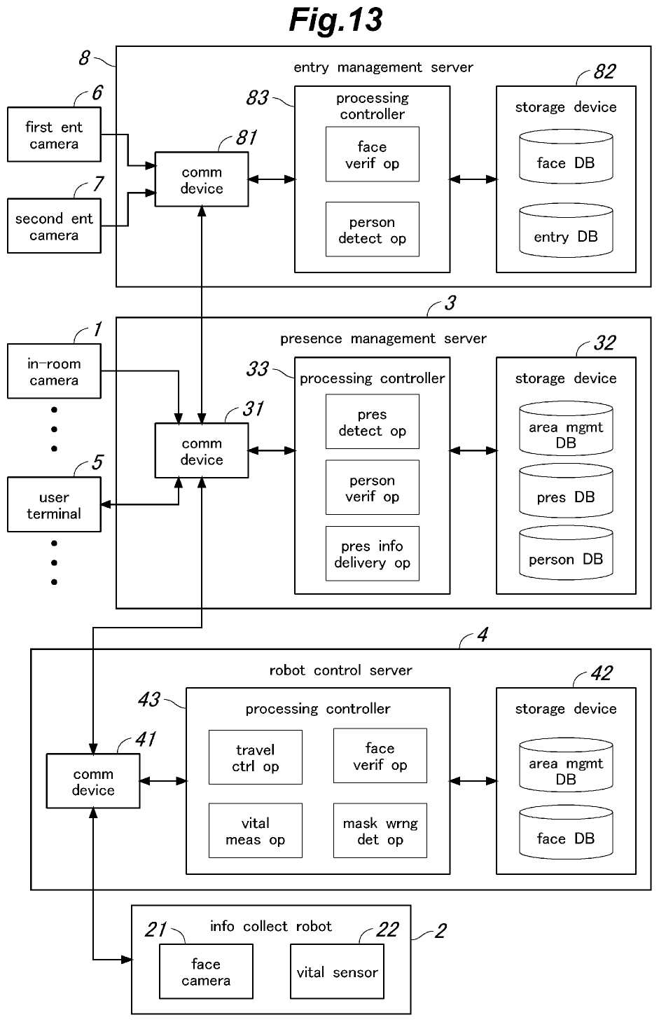

Next, schematic configurations of the entry management server 8, the presence management server 3, and the robot control server 4 of the second embodiment will be described. FIG. 13 is a block diagram showing schematic configurations of the entry management server 8, the presence management server 3, and the robot control server 4.

The entry management server 8 includes a communication device 81, a storage device 82, and a processing controller 83.

The communication device 81 communicates with the first entrance camera 6 and the second entrance camera 7, and receives captured images from the first entrance camera 6 and the second entrance camera 7. The communication device 81 also communicates with the presence management server 3.

The storage device 82 stores programs to be executed by the processing controller 83 and other data. The storage device 82 stores registered information records for a face database. The face database contains face verification information for each person previously registered, which specifically includes information records such as a person ID and face feature data of each person. The storage device 82 also stores registered information records for an entry database. The entry database contains face verification result information, which specifically includes information records such as face verification time, a camera ID, a person ID, and a face verification score, as well as person feature data. Moreover, the storage device 82 temporarily stores images captured by the second entrance camera 7.

The processing controller 83 performs various processing operations by executing programs stored in the storage device 82. In the present embodiment, the processing controller 83 performs the face verification operation, the person detection operation, and other operations.

In the face verification operation (face authentication operation), the processing controller 83 cuts out a person's face from an image captured by the first entrance camera 6 to acquire a face image of an entering person, i.e., a person who is entering the office, and extracts face feature data from the acquired face image. Then, the processing controller 83 acquires face feature data of each registered person from the storage device 82, and compares the face feature data of the entering person with face feature data of each registered person, to thereby identify the entering person.

In the person detection operation, the processing controller 83 detects a person from an image captured by the second entrance camera 7, and cuts out a part of the whole body or upper body of the entering person from the image to thereby acquire a person image of an entering person, i.e., a person who is entering the office, and extracts person feature data from the acquired person image. The processing controller 83 registers the extracted person feature data of the entering person in the entry database of the entry management server 8. A person image may be cut out directly from an image captured by the second entrance camera 7 (an omnidirectional image captured by an omnidirectional camera). In this case, the processing controller 83 may convert an omnidirectional image to a panoramic image, and then cut out a person image from the panoramic image.

The processing controller 83 registers face verification result information (information acquired in the face verification operation) in association with person feature data (data acquired in the person detection operation) in the entry database. In order to avoid performing the face verification operation and the person detection operation on different persons, the processing controller 83 preferably performs the person detection operation using an image captured at the same time as or immediately after capturing an image for face verification. This configuration enables extraction of person feature data of a person from a person image captured during or immediately after the face verification operation for the person.

In the present embodiment, the configuration of the presence management server 3 is the same as that of the first embodiment (see FIG. 5). The processing controller 33 of the presence management server 3 performs the presence detection operation and the presence information delivery operation as in the first embodiment. In the present embodiment, the processing controller 33 further performs the person verification operation.

In the person verification operation, when the presence status of the presence detection area has changed from vacant to present, i.e., when a person takes the seat, the processing controller 33 cuts out a part of the person's whole body or upper body from an image captured by the in-room camera 1 immediately before the person takes a seat to thereby acquire a person image, and extracts person feature data of the person from the person image captured immediately before the person takes the seat. Through the person verification operation, the processing controller 33 associates the detected seated person with an entering person registered in the entry database, to thereby identify the seated person.

In order to acquire a person image of a person immediately before the person takes a seat, the processing controller 33 may acquire an image captured by an in-room camera 1 at a time point of a predetermined number of frames before the detection of a seated person, and acquires a person image from the image captured at the time point slightly before the detection. When the in-room camera 1 is an omnidirectional camera, the processing controller 33 may convert an omnidirectional image to a panoramic image, and acquire a person image from the panoramic image. When extracting person feature data of a person immediately before taking a seat, the processing controller 33 may select, among persons detected in an image captured by an in-room camera 1, a person at a location near the office seat where the seated person is detected, and extract person feature data of the selected person.

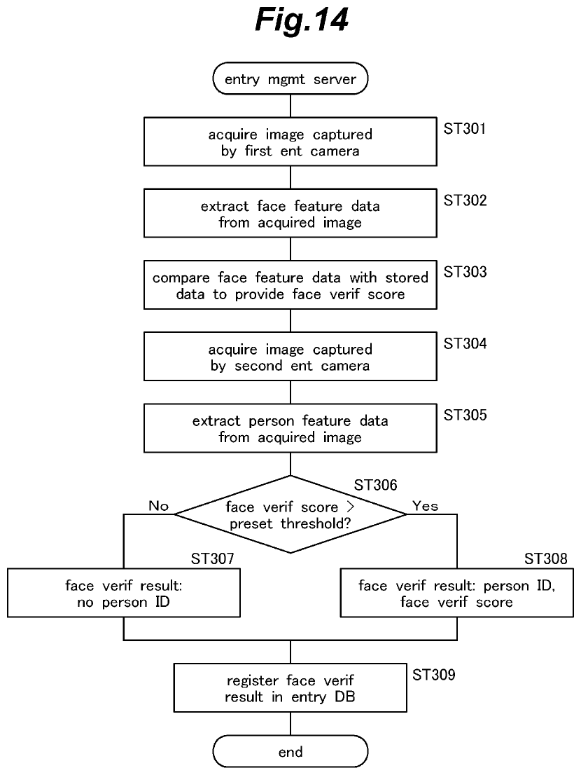

Next, a procedure of operations performed by the entry management server 8 of the second embodiment will be described. FIG. 14 is a flow chart showing a procedure of operations performed by the entry management server 8.

In the entry management server 8, first, the processing controller 83 acquires an image captured by the first entrance camera 6, the image being received by the communication device 81 (ST301). Next, the processing controller 83 extracts face feature data of an entering person, i.e., a person who is entering the office from the image captured by the first entrance camera 6 (ST302). Next, the processing controller 83 acquires face feature data for each registered person from the storage device 82, and compares the face feature data of the entering person with the acquired face feature data for each registered person for verification, to thereby provide a face verification score (ST303).

Next, the processing controller 83 acquires an image captured by the second entrance camera 7, the image being received by the communication device 81 (ST304). In this step, the processing controller 83 acquires an image that was captured by the second entrance camera 7 at the same time as or at a time close to the time when the first entrance camera 6 captured the image. Next, the processing controller 83 extracts person feature data of the entering person from the image captured by the second entrance camera 7 (ST305).

Next, the processing controller 83 determines whether the face verification score is equal to or greater than a predetermined threshold value (face verification score determination) (ST306).

When the face verification score is equal to or greater than the threshold value, i.e., face verification successfully completes (Yes in ST306), the processing controller 83 identifies the person entering the office and generates face verification result information including the person ID and the face verification score (ST308). When the face verification score is less than the threshold value, i.e., face verification completes in failure (No in ST306), the processing controller 83 generates face verification result information including no person ID, assuming that there is no corresponding person (ST307).

Next, the processing controller 83 registers the face verification result information and the person feature data in the entry database (ST309).

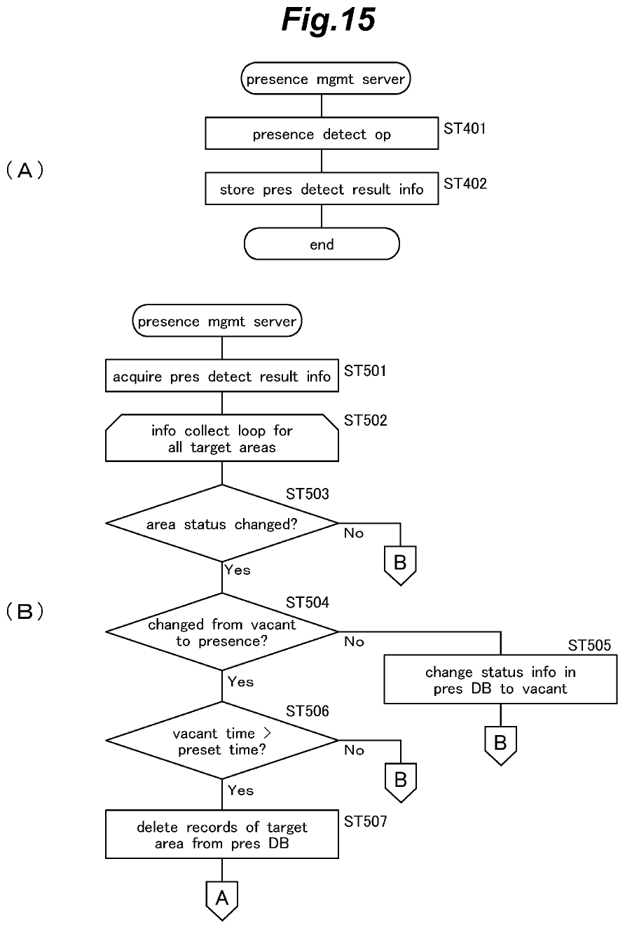

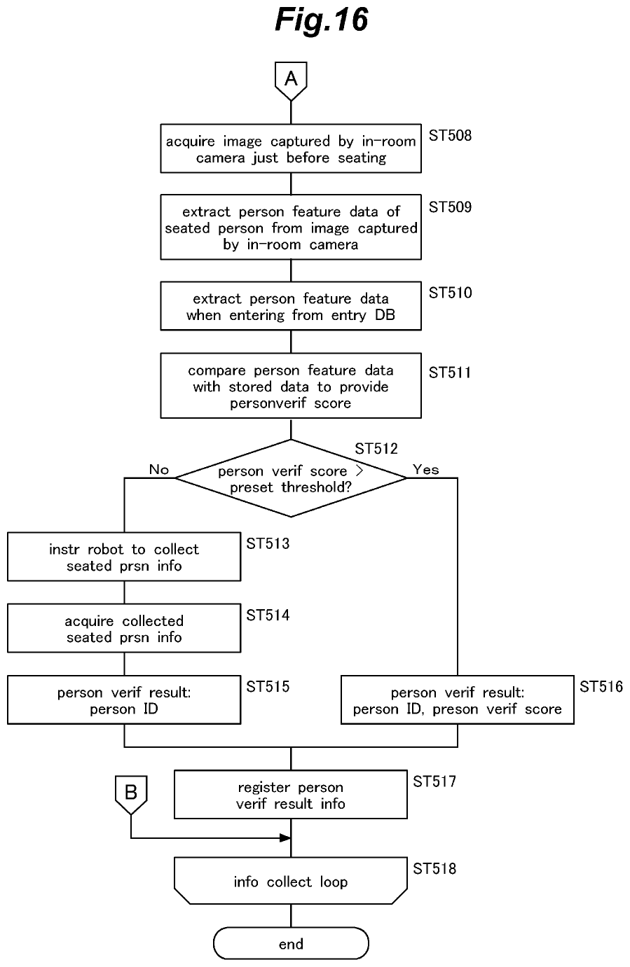

Next, a procedure of operations performed by the presence management server 3 of the second embodiment will be described. FIGS. 15 and 16 are flow charts showing a procedure of operations performed by the presence management server 3.

In the presence management server 3, as shown in FIG. 15(A), the processing controller 33 first performs the presence detection operation (ST401) in the same manner as the first embodiment. Then, the processing controller 33 stores presence detection result information in the storage device 32 (ST402).

In the presence management server 3, as shown in FIG. 15(B) and FIG. 16, the processing controller 33 acquires presence detection result information from the storage device 32 (ST501). Then, the processing controller 33 performs an information collection operation (information collection loop) on each presence detection area for which the presence status of “presence” has been detected (ST502-ST518).

In the information collection operation, the processing controller 33 first determines whether or not the presence status of a presence detection area has changed (ST503).

When determining that the presence status of the presence detection area has changed (Yes in ST503), then the processing controller 33 determines whether or not the presence status of the presence detection area has changed from “vacant (absent)” to “present” (ST504).

When determining that the presence status of the presence detection area has changed from vacant to present, i.e., that a person takes the seat (Yes in ST504), then the processing controller 33 determines whether or not an absence time period, which is a period of time over which the presence status of the presence detection area has been vacant (i.e., no seated person has been present in the presence detection area) before the change in the presence status to “present” (before the detection of the seated person), is equal to or less than a predetermined time period (e.g., three hours) (ST506).

When determining that the absence time period is equal to or less than the predetermined time period, which means that the seated person is likely to be temporarily away from the seat and returns to the seat (No in ST506), the process proceeds to the operation on another presence detection area.

When determining that the absence time period is more than the predetermined time period, which means that the seated person is not likely to be temporarily away from the seat and returns to the seat (Yes in ST506), the processing controller 33 deletes stored person information on the target presence detection area in the presence database (ST507).

Next, the processing controller 33 acquires, from the storage device 42, an image which was captured by an in-room camera 1 immediately before the person's taking the seat (ST508). Then, the processing controller 33 extracts person feature data of the person from the image captured by the in-room camera 1 immediately before the person's taking the seat (ST509).

Next, the processing controller 33 acquires, from the entry management server 8, person feature data of the person extracted from an image captured when the person entered the office and stored in the entry database (ST510). Then, the processing controller 33 compares the person feature data captured at the entrance with the person feature data captured immediately before the person taking the seat, to thereby provide a person verification score (ST511).

Next, the processing controller 33 determines whether or not the person verification score is equal to or more than a predetermined threshold value (ST512).

When the person verification score is equal to or greater than the threshold value, i.e., person verification successfully completes (Yes in ST512), the processing controller 33 identifies the seated person and generates person verification result information including the person ID and the person verification score (ST516).

When the person verification score is less than the threshold value, i.e., person verification completes in failure (No in ST512), the processing controller 33 generates person verification result information including no person ID, assuming that there is no corresponding person (ST513).

Next, the processing controller 33 acquires seated person information on the seated person from the robot control server 4, the seated person information being received by the communication device 31 and including the person ID, vital measurement results, and a result of mask-wearing determination as the face verification results (ST514).

Next, the processing controller 33 generates person verification result information including the person ID (ST515).

Next, the processing controller 33 registers the person verification result information in the presence database (ST517). Then the process proceeds to the operation on another presence detection area. When the person verification successfully completes, after the information on the identified person is stored in the presence database, the processing controller 33 deletes the information on the identified person from the entry database.

When determining that the presence status of the presence detection area has changed from presence to vacant (No in ST504), then the processing controller 33 updates information records of the presence status of the presence detection area in the presence database to “vacant” (ST505). Then, the process proceeds to the operation on another presence detection area.

A procedure of operations performed by the robot control server 4 is same as that of the first embodiment (see FIG. 9).

While specific embodiments of the present invention are described herein for illustrative purposes, the present invention is not limited to those embodiments. It will be understood that various changes, substitutions, additions, and omissions may be made for elements and features of the embodiments without departing from the scope of the invention. In addition, elements and features of the different embodiments may be combined with each other as appropriate to yield an embodiment which is within the scope of the present invention.

### INDUSTRIAL APPLICABILITY

A presence information management system and a presence information management method according to the present invention enable implementation of systems which can be updated and adjusted with less time and effort for adaptation to a change in the office layout or other changes, and which also can efficiently collect information on the person's health, and are useful as a presence information management system and a presence information management method, in which a processing controller manages presence information on a person's presence in an office area.

### Glossary

1 in-room camera (in-area camera)
2 information collection robot
3 presence management server
4 robot control server
5 user terminal
6 first entrance camera
7 second entrance camera
8 entry management server
21 face camera
22 vital sensor
23 speaker
24 travel device
25 control device
31 communication device
32 storage device
33 processing controller
41 communication device
42 storage device
43 processing controller
51 layout drawing
52 person icon
53 word balloon
54 mask icon
55 department group indicating section
56 personal data section
81 communication device
82 storage device
83 processing controller

1. A presence information management system, in which a processing controller manages presence information on a person's presence in an office area, the system comprising:
an in-area camera for capturing an area image of an area within the office area; and
an information collection robot configured to move within the office area,
wherein the information collection robot comprises a face camera for capturing a face image of a seated person in a presence detection area in the office area,
wherein, when detecting a seated person in a presence detection area based on an area image captured by the in-area camera, the processing controller controls the information collection robot such that the information collection robot moves to a location near the presence detection area where the seated person has been detected and captures a face image of the seated person with the face camera, and
wherein, when the face image is captured, the processing controller performs a face verification operation, aiming to verify identity of the seated person, based on the face image captured by the face camera, and when the face verification operation successfully completes, the processing controller generates the presence information associated with the seated person.

2. The presence information management system as claimed in claim 1, wherein the processing controller generates information as to whether the seated person in the presence detection area is wearing a mask or no mask.

3. The presence information management system as claimed in claim 1, wherein the information collection robot is provided with a vital sensor, and
wherein the processing controller causes the information collection robot to measure vital signs of the seated person in the presence detection area by using the vital sensor, thereby acquiring vital information.

4. The presence information management system as claimed in claim 3, wherein the processing controller acquires at least one of a body temperature and a heart rate as the vital information.

5. The presence information management system as claimed in claim 1, wherein, when detecting a seated person in the presence detection area, the processing controller determines whether or not an absence time period, which is a period of time over which no seated person has been present in the presence detection area before the detection of the seated person, is equal to or less than a predetermined time period, and
wherein, when determining that the absence time period is equal to or less than the predetermined time period, the processing controller holds the presence information associated with the detected seated person, and when determining that the absence time period is more than the predetermined time period, the processing controller deletes the presence information associated with the detected seated person.

6. The presence information management system as claimed in claim 1, wherein the processing controller generates a presence map which indicates a position of each seated person in the office area.

7. A presence information management system, in which a processing controller manages presence information on a person's presence in an office area, the system comprising:
an entrance camera for capturing an entrance image of an area in and around an entrance to the office area;
an in-area camera for capturing an area image of an area within the office area; and
an information collection robot configured to move within the office area,
wherein the information collection robot comprises a face camera for capturing a face image of a seated person in a presence detection area in the office area,
wherein the processing controller performs a person identification operation on a person who is entering the office area, to thereby identify the person who has entered the office area,
wherein, when detecting a seated person in a presence detection area based on an area image captured by the in-area camera, the processing controller performs a person verification operation, aiming to verify identity of the seated person, based on an entrance image captured by the entrance camera and the area image captured by the in-area camera, and when the person verification operation successfully completes, the processing controller generates the presence information associated with the seated person,
wherein, when the person verification operation completes in failure, the processing controller controls the information collection robot such that the information collection robot moves to a location near the presence detection area where the seated person has been detected and captures a face image of the seated person with the face camera, and
wherein, when the face image is captured, the processing controller performs a face verification operation, aiming to verify identity of the seated person, based on the face image captured by the face camera, and when the face verification operation successfully completes, the processing controller generates the presence information associated with the seated person.

8. A presence information management method in which a processing controller manages presence information on a person's presence in an office area, the method comprising:
detecting a seated person in a presence detection area based on an area image captured by an in-area camera;
upon the detection, controlling an information collection robot provided with a face camera such that the information collection robot moves to a location near a presence detection area where the seated person has been detected and captures a face image of the seated person with the face camera; and
performing a face verification operation, aiming to verify identity of the seated person, based on the face image captured by the face camera, and when the face verification operation successfully completes, generating the presence information associated with the seated person.

9. (canceled)
